(19) United States
(12) Patent Application Publication
(10) Pub. No.: US 2008/0145435 A1
(43) Pub. Date: Jun. 19, 2008

(54) Composition Comprising a Volatile Solvent, an Organic Filler and a Mineral Filler, and Uses Thereof

(75) Inventors: Audrey Ricard, Paris (FR); Franck Girier Dufournier, Paris (FR); Patricia Lemann, Creteil (FR); Helene Khachikian, Alfortville (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL S.A.

(21) Appl. No.: 11/952,341

(22) Filed: Dec. 7, 2007

Related U.S. Application Data
(63) Continuation of application No. 10/902,936, filed on Aug. 2, 2004, now abandoned.
(60) Provisional application No. 60/493,347, filed on Aug. 8, 2003.

(30) Foreign Application Priority Data
Aug. 1, 2003 (FR) 03 09564

(51) Int. Cl. A61K 9/14; A61K 8/18; A61K 47/04; A61K 33/06; A61Q 19/00

(52) U.S. Cl. 424/489; 424/64; 424/63; 514/770; 514/769; 424/684

(57) ABSTRACT

The disclosure relates to cosmetic compositions, including makeup and care compositions for the skin, comprising at least one fatty phase comprising at least one volatile oil, at least one organic filler and at least one mineral filler, wherein the composition has:
a haze index, (Th−Td)/Th*100, of greater than or equal to 40%, and
a transparency index, Th, of greater than or equal to 70%,
   wherein Th is the hemispheric transmittance of the composition and Td is the direct transmittance of the composition.

This composition may be used to mask wrinkles and other defects of the contour of the lips. It may also be used to modify the visual perception of the volume of the part of the body on which it is applied.

This application claims benefit of U.S. Provisional Application No. 60/493,347, filed Aug. 8, 2003.

The disclosure relates to a cosmetic composition, such as a makeup and/or care composition, that is useful for decreasing or eliminating imperfections of the skin via an optical effect, for example, by diffusing the incident light in several directions.

The compositions disclosed herein may be, for example, in the form of a lipstick or of a foundation, or other types of makeup and/or care compositions. They may allow, for example, the wrinkles and other defects of the contour of the lips to be masked and may result in a modified visual perception of the volume of the part of the body on which they are applied.

Generally, the aim of foundation compositions is to unify the complexion. “Matting” compositions are often used for this purpose, more specifically to help to fade out skin defects such as microreliefs, wrinkles, pores, and variations in skin color. These matting compositions give the skin a matt appearance by diffusing the light at the surface of the skin.

Conventional matting compositions generally contain relatively few fatty substances. Instead, they generally contain a high proportion of powders to adsorb sebum and excess oil of the composition not adsorbed by the skin. Conventional matting powders of natural and synthetic origin include, for example, fillers such as talc, starch, mica, silica, nylon powders, polyethylene powders, poly-beta-alanine, and poly(methyl methacrylates). These types of fillers may have the disadvantage, at high contents, of giving an unnatural appearance to the skin, since they may leave a powdery, or even plastery, whitish-colored deposit. In addition, compositions containing these types of fillers may cause drying out in the long term and also may be difficult to spread.

The present disclosure relates to makeup and care compositions which make it possible to visually mask imperfections of the skin, for example wrinkles, pores, marks, irregular texture, and differences in tone or spots. The compositions of the disclosure may form a translucent film on the skin and thus give the made-up skin a natural appearance.

Unlike certain compositions of the prior art, the compositions of the disclosure may leave no visible white-colored or tinted traces on the skin. The consumer does not need to choose a product tint closest to his or her complexion since the compositions can adapt to any skin type and provide an immediate visual effect of improvement of the uniformity of the skin by masking the defects.

It has been proposed, in U.S. Pat. No. 6,511,672, to combine platelets of alumina treated with iron oxide and platelets of mica coated with silica beads, in order to create a mosaic of color at the microscopic level and to diffuse light so as to produce a screen which makes the lines, wrinkles, deformities and defects of coloration of the skin less visible to the naked eye.

Similarly, U.S. Pat. No. 6,174,533 describes the use of titanium dioxide, zinc oxide or zirconium dioxide with a particle size of between 100 and 300 nm and a refractive index at least equal to 2, for masking wrinkles and other uneven surface textures of the skin.

However, there is a still a need for cosmetic compositions which make it possible to obtain uniform and homogenous skin, such as the complexion, lips, and contour of the lips, which has a natural, radiant, and alive appearance, and at the same time, where such compositions are very comfortable after application to the skin. Additionally, there remains a need for cosmetic compositions which provide a softer visual effect in order to more effectively camouflage the microreliefs of the skin and its disparities in complexion.

The present inventors have discovered, surprisingly, that combining a volatile oil, an organic filler, and a mineral filler results in cosmetic compositions which may more effectively mask imperfections of the skin and make it possible to modify the perception of volume of the part of the body on which they are applied, while at the same time preserving the natural appearance of the skin.

In one aspect, the disclosure provides a cosmetic composition, such as a makeup and/or care composition, comprising at least one fatty phase containing at least one volatile oil, at least one organic filler, and at least one mineral filler, wherein the composition has:

a haze index, (Th−Td)/Th*100, of greater than or equal to 40%, and

a transparency index, Th, of greater than or equal to 70%,

wherein Th is the hemispheric transmittance of the composition and Td is the direct transmittance of the composition.

Such a composition gives the complexion, the lips, the contour of the lips, and more generally the skin, at least one property chosen from greater visual uniformity, greater homogeneity, transparency, and radiance. When applied to the lips, the composition allows the lips to be made smooth via an optical effect, and allows the wrinkles and imperfections of the lips and the contour of the lips to be masked.

In another aspect, the disclosure provides a cosmetic composition, such as a makeup and/or care composition, comprising particles of mica surface-treated with aluminum hydroxide, wherein the composition has:

a haze index, (Th−Td)/Th*100, of greater than or equal to 40%, and

a transparency index, Th, of greater than or equal to 70%,

wherein Th is the hemispheric transmittance of the composition and Td is the direct transmittance of the composition.

The particles of mica are present in this embodiment, for example, in a sufficient amount to obtain a satisfactory haze index and transparency index.

In another aspect, the disclosure provides a cosmetic composition, such as a makeup and/or care composition, comprising coated or uncoated particles of mica and particles of modified or unmodified starch.

The use of at least one mineral filler, of at least one organic filler and of at least one volatile oil in a cosmetic composition, for masking imperfections of the skin and/or modifying the perception of the volume of the part of the body on which the composition is applied, while at the same time preserving a natural appearance of the skin to which it is applied is also provided.

As used herein, the term “fillers” means white or colored, mineral or organic, lamellar or non-lamellar particles, which are solid at room temperature (25° C.) and atmospheric pressure, which do not interact chemically with the other ingredients of the composition, and which are insoluble in the other ingredients, even when the other ingredients are brought to a temperature above room temperature. Crosslinked elastomeric organopolysiloxanes such as those described in EP-A-0 295 886 and U.S. Pat. No. 5,266,321 are not considered fillers as the term is used herein.

As used herein, the term “organic fillers” means fillers containing a single organic chemical compound or fillers containing an organic substrate coated with at least one other organic or mineral compound.

As used herein, the term “mineral fillers” means fillers containing a single mineral chemical compound or fillers containing a mineral substrate coated with at least one other organic or mineral compound.

The haze index, (Th−Td)/Th*100, may be measured according to the protocol described below using a spectrophotometer and an integration sphere, for example, placed behind the sample.

Th is the hemispheric transmittance of the composition: it is defined as the ratio of the light intensity received by the composition and the light intensity released by the composition in all directions,

Td is the direct transmittance of the composition: it is defined as the ratio between the light intensity received by the composition and the light intensity released by the composition in the same axis.

In one embodiment, Th and Td may be measured using a Varian Cary 300 spectrophotometer and a Labsphere brand integration sphere placed behind the sample according to the following protocol: a 20 μm thick film of the composition to be measured is spread onto a quartz crucible slide and then placed in a 37° C. environment for 5 minutes.

To measure the Th, the spectrophotometer is used in diffuse transmission mode, at a wavelength ranging from 400 nm to 700 nm. The machine is set in % T transmission mode, at a scanning speed of 240 nm/min, in “double reverse” mode. A baseline correction is made by measuring an empty slide reference to give the maximum value of the intensity that may be transmitted. The quartz slide containing the film is placed in the measuring compartment and the Th is measured.

The Td is measured using the same spectrophotometer, according to the following protocol. The spectrophotometer is used in direct transmission mode, at a wavelength ranging from 400 nm to 700 nm. The machine is set in % T transmission mode, at a scanning speed of 240 nm/min, in “double” mode. An empty quartz slide is placed in the reference compartment and the quartz slide containing the sample is placed in the measuring compartment and the Td is measured.

The values of Th and Td are the means of each of the spectral values measured.

The higher the value of (Th−Td)/Th*100, the better the haze effect. The higher the Td value, the more transparent the composition.

The haze index of the compositions disclosed herein is greater than or equal to 40%, for example, greater than or equal to 50%.

The transparency index is greater than or equal to 85%, for example, greater than or equal to 95%.

In some embodiments, the at least one organic filler and the at least one mineral filler have, independently of one another, a mean particle size of less than or equal to 50 microns, for example, less than or equal to 30 microns, less than or equal to 15 microns, and less than or equal to 10 microns, such as on the order of 5 or 3 microns.

It is thus possible to obtain a composition which comprises only a few pigments, or even no pigments, but which camouflages the microreliefs and other imperfections of the skin very well. According to one embodiment, the compositions are free of pigments and/or free of pearlescent agents.

In some embodiments, the at least one mineral filler comprises a material chosen from, for example, talc, mica, silica, kaolin, hollow microspheres of silica, glass microcapsules, titanium oxide, iron oxide, and zinc oxide. The mineral filler may optionally be coated and may comprise a mica substrate coated with alumina, titanium dioxide, silica, aluminum oxide and/or barium sulfate, such as TiO₂/silica, mica/TiO₂, silica/TiO₂, and the complex of silica and of TiO₂/TiO₂. The filler may be in lamellar or non-lamellar form.

In some embodiments, the at least one mineral filler is chosen from, for example, silica, talc, mica and composites containing mica, composites of silica and of titanium dioxide, composites of silica and of zinc oxide, and mixtures thereof.

Mineral fillers that may be used in the compositions disclosed herein include, but are not limited to:
titanium oxide covered with silica, such as Flonac TS 40 C (Eckart),
silica microbeads with a mean particle size ranging from 3 to 15 microns, such as Silica Beads SB 150 (Miyoshi), or Sunsphere H31 silica microbeads with a mean particle size equal to 3 microns (Asahi Glass),
platelets of silica with a mean particle size equal to 1.5 microns, for example Chemiceler or Finesil F-80 (Sumitomo),
mica platelets covered with aluminium hydroxide microbeads in a weight ratio of 60/40 or of 65/35, such as Excel Mica JP-1 or Excel Mica JP-2 (Miyoshi),
silica coated with titanium dioxide and with porous silica, with a mean particle size equal, for example, to 0.6 microns, such as ACS-0050510 (Catalysts and Chemicals), wherein the silica/titanium dioxide/porous silica proportion is 85/5/10,
mica covered with barium sulfate and with titanium oxide, such as Naturaleaf (Merck), wherein the mica/barium sulfate/titanium oxide proportion is 66/22/12,
a complex of silica and of cerium oxide coated with amorphous silica, with a mean particle size of between 1 and 10 microns, such as Ceriguard SC,
a complex of silica and of titanium oxide coated with polyhydrogenomethylsiloxane, for example, in the proportions 93/5/2,
sericite platelets covered with titanium oxide, with alumina, and with silica, such as, for example, Coverleaf AF-20121M (Catalysts and Chemicals), wherein the mean particle size ranges from 5 to 10 μm and the sericite/titanium oxide/alumina/silica proportion is 67/5/18/10.

In some embodiments, the at least one mineral filler is chosen from composites of mica and of a metal hydroxide. The at least one mineral filler may comprise mica covered with aluminum hydroxide mica beads and have a mean particle size equal to 10 microns (plus or minus 2 microns).

As used herein, the mean particle size (or granulometry), D50, is defined as the size given, by the statistical mean particle size distribution, of half the population.

In some embodiments, the at least one mineral filler is present in the composition in a in an amount ranging from 0.1% to 40%, for example, from 1% to 15%, or from 2% to 10%, such as 5%, by weight relative to the total weight of the composition.

Suitable organic fillers include, but are not limited to, fillers made of polyamide powders, for example, Nylon® powders, poly-β-alanine powders, polyethylene powders, polytetrafluoroethylene powders, lauroyllysine, starch, hollow polymer micro-spheres such as those of polyvinylidene chloride/acrylonitrile, e.g., Expancel®, acrylic acid copolymers, e.g., Polytrap®, and silicone resin microbeads, e.g., Tospearl.

In some embodiments, the at least one organic filler is starch, such as modified starch, for example, the starch crosslinked with octenylsuccinic anhydride sold as Dry Flo Plus (28-1160) (National Starch).

In some embodiments, the at least one organic filler is present in the composition in an amount ranging from 0.1% to 40%, for example, from 3% to 20%, such as from 8% to 15%, by weight relative to the total weight of the composition.

The at least one organic filler and the at least one mineral filler may have a refractive index of greater than 1, for example, ranging from 1.25 to 1.9 or from 1.45 to 1.55.

The at least one mineral filler and the at least one organic filler may be present in the composition in a mass ratio ranging from 1:1 to 1:3.

According to some embodiments, the at least one mineral filler is lamellar when the organic filler is spherical, and vice versa. As used herein, “in lamellar form” means in plateform; particles for which the ratio of the largest dimension to the thickness is greater than or equal to 5, such as greater than or equal to 10, or greater than or equal to 20. As used herein, “spherical” means a spherical form, such as in the form of beads, wherein the mean particle size ranges from 1 to 15 microns, such as 10 microns.

The composition according to the disclosure may comprise less than 30% of fillers, for example, less than 20%.

In another aspect, a cosmetic composition is provided, such as a makeup and/or care composition, comprising at least one mineral filler chosen from composites of an inorganic silicate and of a metal hydroxide.

In certain embodiments, the composition has

a haze index, (Th−Td)/Th*100, of greater than or equal to 40%, and

a transparency index, Th, of greater than or equal to 70%,

wherein Th is the hemispheric transmittance of the composition and Td is the direct transmittance of the composition.

The inorganic silicate may be natural or synthetic mica. The mica may be chosen from muscovite, phlogopite, biotite, sericite, lepidolite and paragonite micas, synthetic micas, and mixtures thereof.

The metal hydroxide may be a metal hydroxide chosen from, for example, Ag, Au, Cu, Al, Ni, Sn, Mg, Cr, Mo, Ti, Pt, Va, Rb, W, Zn, Ge, Te and Se, and alloys thereof. One such metal hydroxide is aluminum hydroxide.

According to some embodiments, the mineral filler is a composite comprising particles of inorganic silicate, surface-covered with a metal hydroxide. The inorganic silicate may be in lamellar form or in platelet form. As used herein, the term “in platelet form” means particles for which the ratio of the largest dimension to the thickness is greater than or equal to 5, 10, or even 20.

The metal hydroxide may be in spherical form, such as in the form of beads, for example, with a mean particle size ranging from 1 to 15 microns, e.g., on the order of 10 microns.

The proportion by mass of the inorganic silicate to the metal hydroxide may range from 50/50 to 80/20, such as from 60/40 to 70/30, or equal to about 65/35.

The beads of metal hydroxide may be beads of aluminum hydroxide and the composition may contain optionally modified starch.

In some embodiments, the composition comprises at least one volatile oil.

As used herein, the term “volatile oil” means any nonaqueous medium capable of evaporating on contact with the skin, at room temperature (25° C.) and atmospheric pressure (760 mm Hg). The volatile cosmetic oil or oils, which are liquid at room temperature, may have a vapor pressure, measured at room temperature and atmospheric pressure, ranging from 10⁻³ mm to 300 mm of Hg (0.266 Pa to 40,000 Pa), for example, from 0.02 mm to 300 mm of Hg (2.66 Pa to 40,000 Pa), and from 0.1 mm to 90 mm of Hg (13 Pa to 12,000 Pa).

The volatile oils may be hydrocarbon oils, silicone oils optionally containing alkyl or alkoxy groups which are pendant or at the end of the silicone chain, or a mixture of these oils.

The volatile oils may be cosmetic oils chosen from oils which do not have a flash point, oils which have a flash point ranging from 40° C. to 100° C., and mixtures thereof. In addition, the oils may have a boiling point at atmospheric pressure of less than or equal to 220° C. or 210° C., e.g., ranging from 110° C. to 210° C. In some embodiments, these volatile oils are not monoalcohols containing at least 7 carbon atoms.

Volatile oils which may be used include, but are not limited to, linear or cyclic silicone oils having a viscosity at room temperature of less than 8 cSt and having from 2 to 7 silicon atoms, optionally comprising alkyl and/or alkoxy groups having from 1 to 10 carbon atoms. Volatile silicone oils include, but are not limited to, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

Other volatile oils which may be used include volatile hydrocarbon-based oils having from 8 to 16 carbon atoms, and mixtures thereof, such as branched C₈-C₁₆ alkanes, e.g., C₈-C₁₆ isoalkanes (also called isoparaffins), isododecane, isodecane or isohexadecane and, for example, oils sold under the trade names Isopars or Permethyls, branched C₈-C₁₆ esters, such as isohexyl neopentanoate, and mixtures thereof.

Specific volatile oils which may be used include isododecane (Permethyls 99 A), C₈-C₁₆ isoparaffins such as Isopar L, E, G or H, and mixtures thereof, optionally combined with decamethyltetrasiloxane or with cyclopentasiloxane.

Volatile fluoro oils may also be used.

The volatile oils may comprise from 5% to 80% of the total weight of the composition, for example, from 10% to 30%.

The compositions may further comprise at least one non-volatile oil.

As used herein, the term “non-volatile oil” means a fatty substance that is liquid at room temperature (25° C.) and that does not evaporate at that temperature.

Non-volatile oils include, but are not limited to:

poly(C₁-C₂₀)alkylsiloxanes such as those containing trimethylsilyl end groups, which may have a viscosity of less than 0.06 m²/s, e.g., oils made up of linear polydimethylsiloxanes and alkylmethylpolysiloxanes such as cetyldimethicone (CTFA name);

silicones modified with optionally fluorinated aliphatic and/or aromatic groups, or with functional groups such as hydroxyl, thiol and/or amino groups;

phenylsilicone oils, such as phenyl trimethicones;

oils of plant or mineral origin, such as liquid paraffin, liquid petroleum jelly, perhydrosqualene, lanolin, apricot oil, wheatgerm oil, sweet almond oil, beauty-leaf oil, sesame seed oil, macadamia oil, grapeseed oil, rapeseed oil, coconut oil, groundnut oil, sunflower oil, palm oil, castor oil, avocado oil, jojoba oil, olive oil, cereal germ oil, the liquid fraction of shea buffer, fatty acid esters of polyols (such as liquid triglycerides), isopropyl myristate, miglyol oil, isostearyl neopentanoate, 2-ethylhexyl palmitate, castor oil, tributyl acetyl citrate, alcohols (such as 2-octyldodecanol), acetylglycerides, octanoates, decanoates or ricinoleates of alcohols or of polyalcohols, fatty acid triglycerides, glycerides, fluoro oils, and perfluoro oils;

amide compounds, such as those described in patent application PCT/FR98/01077 (WO 98/56754), for example, N-neopentanoyl-2-octyldodecylamine, N-neopentanoyl-2-butyloctylamine, N-(3,5,5-trimethylhexanoyl)-2-octyldodecylamine, and N-(3,5,5-trimethylhexanoyl)-2-butyloctylamine; and

mixtures thereof.

In one embodiment, the at least one non-volatile oil of the fatty phase of the composition according to the disclosure comprises at least one oil chosen from hydrogenated polyisobutene, arachidyl propionate, octyldodecyl neopentanoate, polybutene, dimethicones and octyldodecanol, and mixtures thereof.

The at least one non-volatile oil may be present in an amount ranging from 1% to 85%, such as from 5% to 60%, from 10% to 50%, or even from 20% to 40% by weight relative to the total weight of the composition.

The composition may also comprise at least one wax and/or at least one pasty compound, for example, when it is in the form of a stick.

As used herein, the term “pasty compound” means a lipophilic fatty compound with a reversible solid/liquid change of state, which has an anisotropic crystal organization in the solid state, and which has a liquid fraction and a solid fraction at 23° C. The pasty compound may have a hardness at 20° C. ranging from 0.001 MPa to 0.5 MPa, e.g., from 0.002 MPa to 0.4 MPa.

The hardness may be measured by penetrating a probe into a sample of the compound and using a texture analyzer (for example the TA-XT21 machine from Rheo) equipped with a stainless-steel 2 mm diameter cylinder. The hardness measurement is performed at 20° C. at the center of 5 samples. The cylinder is introduced into each sample at a prespeed of 1 mm/s and then at a measuring speed of 0.1 mm/s with a penetration depth of 0.3 mm. The maximum peak recorded is the hardness value.

At 23° C., the pasty compound is in the form of a liquid fraction and a solid fraction so the starting melting point of the pasty compound is less than 23° C. The liquid fraction of the pasty compound measured at 23° C. represents from 9% to 97% by weight of the compound, such as from 15% to 85% or from 40% to 85% by weight.

The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound is equal to the area under the curve of a thermogram obtained using a differential scanning calorimeter (DSC), such as aMDSC 2920® calorimeter (TA Instrument), with a temperature rise of 5° C. or 10° C. per minute, following standard ISO 11357-3:1999. The heat of fusion of the pasty compound is the amount of energy required to make the compound change from the solid state to the liquid state and is expressed in J/g.

The heat of fusion consumed at 23° C. is the amount of energy absorbed by the sample to change from the solid state to the state that it has at 23° C., having a liquid fraction and a solid fraction.

The liquid fraction of the pasty compound, measured at 32° C., may comprise from 30% to 100% by weight of the compound, such as from 80% to 100% or even 90% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

The pasty compound may be a synthetic compounds or a compound of plant origin. A pasty compound may also be obtained synthetically from starting materials of plant origin.

Pasty compounds include, but are not limited to:

lanolin and lanolin derivatives;

polymeric or non-polymeric silicone compounds;

polymeric or non-polymeric fluoro compounds;

vinyl polymers, such as:
olefin homopolymers,
olefin copolymers,
hydrogenated diene homopolymers and copolymers,
linear or branched oligomers, which are homopolymers or copolymers of alkyl (meth)acrylates optionally having a C₈-C₃₀ alkyl group,
oligomers, which are homopolymers and copolymers of vinyl esters having C₈-C₃₀ alkyl groups, and
oligomers, which are homopolymers and copolymers of vinyl ethers having C₈-C₃₀ alkyl groups;
liposoluble polyethers resulting from the polyetherification between one or more C₂-C₁₀₀, such as C₂-C₅₀ diols;
esters; and
   mixtures thereof.

The pasty compound may comprise from 1% to 99%, such as from 1% to 60%, 2% to 30%, or 5% to 15% by weight of the composition.

Waxes that may be used include, but are not limited to: waxes of animal, plant, mineral or synthetic origin, such as microcrystalline waxes, paraffin, petrolatum, petroleum jelly, ozokerite or montan wax; beeswax and lanolins and lanolin derivatives; candelilla wax, ouricury wax, carnauba wax, Japan wax, cocoa butter, cork fibre wax or sugarcane wax; hydrogenated oils that are solid at 25° C., ozokerites, fatty esters and glycerides that are solid at 25° C.; polyethylene waxes and waxes obtained by Fischer-Tropsch synthesis; hydrogenated oils that are solid at 25° C.; fatty esters that are solid at 25° C.; silicone waxes; fluoro waxes; and mixtures thereof.

The compositions may also contain additional fillers other than those described above.

These additional fillers may be present in the composition in an amount ranging from 0% to 20% by weight, such as from 2% to 15% by weight, relative to the total weight of the composition. In certain embodiments, the composition is free of such additional fillers.

The compositions may also comprise dyes, for instance water-soluble or liposoluble dyes known in the art, such as the disodium salt of ponceau, the disodium salt of alizarine green, quinoline yellow, the trisodium salt of amaranth, the disodium salt of tartrazine, the monosodium salt of rhodamine, the disodium salt of fuchsin, xanthophyll, Red Sudan III (CTFA name: R&D red 17), lutein, quinizarine green (CTFA name: DC green 6), Alizurol SS violet (CTFA name: DC violet No. 2), carotenoid derivatives such as lycopene, beta-carotene, bixin, capsanthin, and/or mixtures thereof.

The compositions may comprise a cosmetically and/or physiologically acceptable medium, i.e., a medium compatible with the skin, which includes any area of the skin on the body and on the face.

Such compositions are generally in fluid or solid form, consequently, such compositions may be in fluid form, in pasty form, in semi-solid form and in solid form; e.g., as a stick. Such compositions may be in the form of a foundation, for example in fluid form, in a pot, or as a stick; in the form of a lip paint, which may be in pasty form; or of lipsticks, which may be in semi-solid form (gloss); or of lipsticks in solid form, as a stick.

The compositions may be in the form of a makeup stick, such as an eyeshadow stick, a foundation stick, a stick for concealing rings under the eyes, or a lipstick.

The compositions may optionally comprise an aqueous phase, which may comprise water, a floral water such as cornflower water and/or a mineral water such as eau de Vittel, eau de Lucas or eau de La Roche Posay, and/or a spring water.

The compositions of the disclosure may thus be in the form of an anhydrous composition or of an oil-in-water (O/W) emulsion, a water-in-oil (W/O) emulsion, a multiple emulsion, or a multiphase solution. They may also be in the form of a vesicular dispersion, for example in the form of an oil phase dispersed in an aqueous phase and stabilized by liposomes.

When the compositions are emulsions, they may comprise amphiphilic compounds, i.e., compounds comprising both a lipophilic component (apolar component) and a hydrophilic component (polar component) and able to adsorb onto a surface or an interface. Such compounds include, for example, surfactants and co-surfactants.

Oil-in-water surfactants include, but are not limited to: stearic acid combined with triethanolamine, a mixture of glyceryl monostearate and distearate, sodium lauroyl sarcosinate, cetearyl glucoside, PEG-40 stearate, sorbitan tristearate, sorbitan stearate, polysorbate 60, a sorbitan stearate/sucrose cocoate mixture, a glyceryl stearate/PEG-100 stearate mixture, PEG-400, glyceryl stearate, and a PEG-6/PEG-32/glycyl stearate mixture (CTFA names). Water-in-oil surfactants include, but are not limited to: a polyglyceryl-4 isostearate/cetyldimethicone copolyol/hexyl laurate mixture and a mineral oil/petrolatum/ozokerite/glyceryl oleate/lanolin alcohol mixture.

In some embodiments, the compositions are in the form of W/O emulsions.

The compositions may also comprise any additive known in the art, such as antioxidants, UV-screening agents, dyes, fragrances, essential oils, preserving agents, cosmetic active agents, moisturizers, vitamins, spingolipids, liposoluble polymers, such as hydrocarbon-based polymers, for example, polybutene, polyalkenes, polyacrylates and silicone polymers compatible with the fatty substances, and/or mixtures thereof.

The additives may be present in the compositions in an amount ranging from 0% to 15% by weight relative to the total weight of the composition.

In some embodiments, the composition is anhydrous and contains less than 5% of water added during preparation, present in the composition as an impurity or adsorbed by the composition. The anhydrous compositions may contain less than 3% of water, or even less than 1%.

Of course, those skilled in the art will take care to choose this or these optional additional compound or compounds, and/or the amount thereof, in such a way that the advantageous properties of the composition according to the disclosure are not, or are not substantially, altered by the envisaged addition.

The compositions may be in the form of a cosmetic product, such as in the form of a makeup product, e.g., a foundation, a product for concealing rings under the eyes, a product for the lips, a makeup product for the body, or alternatively a care product for the body and/or the face.

The compositions according to the disclosure may be prepared using conventional methods of preparation known to those skilled in the art.

The invention is illustrated in greater detail by the example described below. Other than in the example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

The following example illustrates the disclosure. In this examples, the proportions indicated are percentages by weight.

### EXAMPLE 1

### Smoothing Composition for the Lips

| | |
|---|---|
| Hydrogenated polyisobutene (NOF Corp.) | 10% |
| Arachidyl propionate (Alzo) | 1.8% |
| Octyldodecyl neopentanoate (Bernel) | 1.3% |
| Disteardimonium hectorite (Elementis) | 0.3% |
| Mica coated with aluminium hydroxide | 5% |
| microbeads (Excel Mica JP-2 ® from | |
| Miyoshi) | |
| Starch crosslinked with octenylsuccinic | 10% |
| anhydride (Dry Flo Plus (28-1160) ® | |
| from National Starch) | |
| Polybutene | 4.9% |
| Polyethylene wax | 15.8% |
| Dimethicone (DC 200-ScSt) | 17.3% |
| Decamethyltetrasiloxane (DC 200-1.5 cSt) | 22% |
| Octyldodecanol | 3.2% |
| Oxypropylenated lanolin wax (5 PO) (Cognis) | 8.4% |

What is claimed is:

1. A cosmetic composition comprising at least one fatty phase comprising at least one volatile oil, at least one organic filler and at least one mineral filler,
wherein the composition has
a haze index (Th−Td)/Th*100 of greater than or equal to 40%, and
a transparency index Th of greater than or equal to 70%,
wherein Th is the hemispheric transmittance of the composition and Td is the direct transmittance of the composition.

2. The composition according to claim 1, wherein the composition is a makeup and/or care composition.

3. The composition according to claim 1, wherein the composition is anhydrous.

4. The composition according to claim 2, wherein the composition is the form of a cosmetic product.

5. The composition according to claim 4, wherein the composition is the form of a makeup product.

6. The composition according to claim 5, wherein the composition is the form of a foundation, a product for concealing rings under the eyes, a makeup product for the body, or a care product for the body and/or the face.

7. The composition according to claim 1, wherein the composition is in the form of a makeup stick.

8. The composition according to claim 7, wherein the composition is in the form of an eyeshadow stick, a foundation stick, a stick for concealing rings under the eyes, or a lipstick.

9. The composition according to claim 1, wherein the haze index is greater than or equal to 50%.

10. The composition according to claim 1, wherein the transparency index is greater than or equal to 85%.

11. The composition according to claim 10, wherein the transparency index is greater than or equal to 95%.

12. The composition according to claim 1, wherein the at least one mineral filler and the at least one organic filler each have, independently of one another, a mean particle size of less than or equal to 50 microns.

13. The composition according to claim 12, wherein the mean particle size is less than or equal to 30 microns.

14. The composition according to claim 13, wherein the mean particle size is less than or equal to 15 microns.

15. The composition according to claim 14, wherein the mean particle size is less than or equal to 10 microns.

16. The composition according to claim 1, wherein the at least one mineral filler is lamellar when the at least one organic filler is spherical, and wherein the at least one mineral filler is spherical when the at least one organic filler is lamellar.

17. The composition according claim 1, wherein the at least one mineral filler is chosen from silica, talc, mica, composites comprising mica, composites of silica and of titanium dioxide, composites of silica and of zinc oxide, and mixtures thereof.

18. The composition according to claim 1, wherein the at least one mineral filler is chosen from:
titanium oxide covered with silica,
silica microbeads with a mean particle size ranging from 3 to 15 microns,
platelets of silica having a mean particle size of 1.5 microns,
silica coated with titanium dioxide and porous silica,
mica platelets covered with aluminium hydroxide microbeads in a weight ratio of 60/40 or of 65/35,
mica covered with barium sulfate and with titanium oxide,
a complex of silica and of cerium oxide coated with amorphous silica and having a mean particle size between 1 and 10 microns,
a complex of silica and of titanium oxide coated with polyhydrogenomethylsiloxane, sericite platelets covered with titanium oxide, with alumina and with silica, and mixtures thereof.

19. The composition according to claim 18, wherein the at least one mineral filler is silica coated with titanium dioxide and porous silica having a mean particle size equal to 0.6 microns.

20. The composition according to claim 18, wherein the at least one mineral filler is silica coated with titanium dioxide and porous silica in a proportion of silica/titanium dioxide/porous silica of 85/5/10.

21. The composition according to claim 18, wherein the at least one mineral filler is mica covered with barium sulfate and with titanium oxide in a proportion of mica/barium sulfate/titanium oxide of 66/22/12.

22. The composition according to claim 1, wherein the at least one mineral filler is chosen from composites of mica and of a metal hydroxide.

23. The composition according to claim 22, wherein the metal hydroxide is aluminium hydroxide.

24. The composition according to claim 1, wherein the at least one organic filler is chosen from polyethylene powders, starch powders, modified starch powders, nylon powders and styrene/acrylic copolymer powders, and mixtures thereof.

25. The composition according to claim 24, wherein the at least one organic filler is starch crosslinked with octenylsuccinic anhydride.

26. The composition according to claim 1, wherein the at least one mineral filler is mica or a composite of mica with a mean particle size of less than or equal to 50 microns.

27. The composition according to claim 26, wherein the at least one mineral filler is mica or a composite of mica with a mean particle size of less than or equal to 10 microns.

28. The composition according to claim 27, wherein the at least one mineral filler is mica or a composite of mica with a mean particle size of less than or equal to 5 microns.

29. The composition according to claim 1, wherein the at least one organic filler is starch with a mean particle size of less than or equal to 50 microns.

30. The composition according to claim 29, wherein the at least one organic filler is starch with a mean particle size of less than or equal to 10 microns.

31. The composition according to claim 30, wherein the at least one organic filler is starch with a mean particle size of less than or equal to 5 microns.

32. The composition according to claim 1, wherein the at least one organic filler is present in an amount ranging from 0.1% to 40% by weight relative to the total weight of the composition.

33. The composition according to claim 32, wherein the at least one organic filler is present in an amount ranging from 3% to 20% by weight relative to the total weight of the composition.

34. The composition according to claim 33, wherein the at least one organic filler is present in an amount ranging from 8% to 15% by weight relative to the total weight of the composition.

35. The composition according to claim 1, wherein the at least one mineral filler is present in an amount ranging from 0.1% to 40% by weight relative to the total weight of the composition.

36. The composition according to claim 35, wherein the at least one mineral filler is present in an amount ranging from 1% to 15% by weight relative to the total weight of the composition.

37. The composition according to claim 36, wherein the at least one mineral filler is present in an amount ranging from 2% to 10% by weight relative to the total weight of the composition.

38. The composition according to claim 37, wherein the at least one mineral filler is present in an amount of 5% by weight relative to the total weight of the composition.

39. The composition according to claim 1, wherein the at least one fatty phase further comprises at least one non-volatile oil.

40. The composition according to claim 39, wherein the at least one non-volatile oil is chosen from hydrogenated polyisobutene, arachidyl propionate, octyldodecyl neopentanoate, polybutene, dimethicones, octyldodecanol, and mixtures thereof.

41. The composition according to claim 39, wherein the at least one non-volatile oil is present in an amount ranging from 1% to 85% by weight relative to the total weight of the composition.

42. The composition according to claim 41, wherein the at least one non-volatile oil is present in an amount ranging from 5% to 60% by weight relative to the total weight of the composition.

43. The composition according to claim 42, wherein the at least one non-volatile oil is present in an amount ranging from 10% to 50% by weight relative to the total weight of the composition.

44. The composition according to claim 43, wherein the at least one non-volatile oil is present in an amount ranging from 20% to 40% by weight relative to the total weight of the composition.

45. The composition according to claim 1, further comprising an aqueous phase.

46. The composition according to claim 1, wherein the composition is in a fluid or a solid form.

47. The composition according to claim 1, wherein the composition is in a pasty or in semi-solid form.

48. A method of masking wrinkles and defects of the skin on the face or of the contour of the lips comprising applying a composition comprising at least one mineral filler, at least one organic filler and at least one volatile oil, to the skin on the face or the lips in an amount effective to mask the wrinkles.

49. A method of modifying the visual perception of the volume of a part of the body comprising applying a composition comprising at least one mineral filler, at least one organic filler and at least one volatile oil to the part of the body in an amount effective to modify the visual perception.

50. A cosmetic composition comprising at least one mineral filler chosen from composites of an inorganic silicate and of a metal hydroxide.

51. The cosmetic composition according to claim 50, wherein the composition is a makeup and/or care composition.

52. The composition according to claim 50, wherein the inorganic silicate is natural or synthetic mica.

53. The composition according to claim 50, wherein the metal hydroxide is aluminum hydroxide.

54. The composition according to claim 50, wherein the at least one mineral filler comprises particles of inorganic silicate, surface-covered with a metal hydroxide.

55. The composition according to claim 54, wherein the inorganic silicate is in lamellar form.

56. The composition according to claim 50, wherein the metal hydroxide is in the form of beads.

57. The composition according to claim 56, wherein the beads have a mean particle size ranging from 1 to 15 microns.

58. The composition according to claim 57, wherein the beads have a mean particle size of 10 microns.

59. The composition according to claim 50, wherein the proportion by mass of the inorganic silicate to the metal hydroxide ranges from 50/50 to 80/20.

60. The composition according to claim 59, wherein the proportion by mass is from 60/40 to 70/30.

61. The composition according to claim 60, wherein the proportion by mass is equal to 65/35.

62. The composition according to claim 50, wherein the composition has
a haze index (Th−Td)/Th*100 of greater than or equal to 40%, and
a transparency index Th of greater than or equal to 70%,
wherein Th is a hemispheric transmittance of the composition and Td is a direct transmittance of the composition.

63. The composition according to claim 50, further comprising starch.

64. The composition according to claim 63, wherein the starch is modified.

65. A cosmetic composition comprising at least one fatty phase comprising at least one volatile oil, at least one organic filler and at least one mineral filler, wherein the composition is free of pigments and of pearlescent agents, and has
a haze index (Th−Td)/Th*100 of greater than or equal to 40%, and
a transparency index Th of greater than or equal to 70%,
wherein Th is the hemispheric transmittance of the composition and Td is the direct transmittance of the composition.
